(19) United States
(12) Patent Application Publication
(10) Pub. No.: US 2025/0032417 A1
(43) Pub. Date: Jan. 30, 2025

(54) LYOPHILIZED METOLAZONE FORMULATION FOR PARENTERAL ADMINISTRATION

(71) Applicant: Hyloris Developments SA, Liège (BE)

(72) Inventor: Atul Patil, Liège (BE)

(21) Appl. No.: 18/775,374

(22) Filed: Jul. 17, 2024

Related U.S. Application Data
(60) Provisional application No. 63/515,327, filed on Jul. 24, 2023.

(51) Int. Cl. A61K 9/19; A61K 31/517; A61K 47/10; A61K 47/26

(52) U.S. Cl. CPC A61K 9/19; A61K 31/517; A61K 47/10; A61K 47/26

(57) ABSTRACT

The invention relates to a novel metolazone lyophilizate composition and a pharmaceutically acceptable diluent that can be used to reconstitute the metolazone lyophilizate for parenteral administration. The invention also relates to a novel metolazone pre-lyophilizate composition as well as uses of the reconstituted metolazone lyophilizate.

### CROSS-REFERENCE TO RELATED APPLICATIONS

This application claims priority to U.S. Provisional Application No. 63/515,327, filed 24 Jul. 2023, the disclosure of which is incorporated herein by reference in its entirety.

### TECHNICAL FIELD

The present invention is situated in the field of pharmaceutical compositions and medical uses of pharmaceutical compositions. The present invention also relates to the manufacturing of pharmaceutical compositions. The active ingredient concerned is metolazone. The invention is advantageous as it provides a stable metolazone formulation suitable for parenteral administration where hereto only metolazone tablets are commercially available. The invention has for an effect that patients with a metolazone-responsive disease can be treated effectively even if unconscious or even if having problems swallowing.

### BACKGROUND OF THE INVENTION

Metolazone is a well-known active ingredient and is commercially available in the form of 2.5 mg, 5 mg, and 10 mg tablets. Its chemical name is 7-chloro-1,2,3,4-tetrahydro-2-methyl-3-(2-methylphenyl)-4-oxo-6-quinazoline sulfonamide (formula I, below). It is poorly soluble in water 2.4×10-5 g/mL at 25° C. and in ethanol 9×10⁻³ g/mL at 25° C.

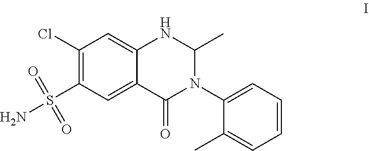 [SMILES: Cc1ccccc1N1C(=O)c2cc(S(N)(=O)=O)c(Cl)cc2NC1C]

Metolazone is a quinazoline diuretic used to reduce the swelling and fluid retention caused by congestive heart failure or chronic kidney disease. Metolazone is used to alleviate salt and water retention, called edema. Metolazone is used in congestive heart failure and to treat hypertension.

There is no injectable form of metolazone commercially available mainly due to its extremely low solubility and instability in solution (e.g., it degrades in solution making long-term storage difficult, if not impossible). Because there is no injectable form of metolazone available, there is an unmet medical need for patients who cannot take metolazone orally or where administration or titration of the metolazone dose is critical to achieve maximum effect or where a fast onset of action is required.

U.S. Pat. No. 5,124,152 describes parenteral formulations of metolazone including an aqueous solution comprising 0.1-8 mg/mL metolazone, 5-15% w/v of 95% ethanol, 0-3% v/v benzyl alcohol, and a cosolvent selected from 30-65 w/v propylene glycol, 25-50% v/v polyethylene glycol 300 or 25-50% v/v polyethylene glycol 400. It has now been found that the use of any of these cosolvents leads to degradation products on storage. Thus, these formulations lack long-term stability.

The liquid metolazone formulations mentioned in U.S. Pat. No. 5,124,152 were proposed as an alternative to a lyophilized solid prepared from the sodium salt of metolazone and sodium hydroxide, which on reconstitution with water provided a pH of about 11. This is too high for administration to a patient.

In U.S. Pat. Nos. 5,633,240, 5,684,009, and 5,814,623, liquid metolazone formulations for parenteral administration were proposed using an aqueous buffer system of pH 10.5 to 12.5, which is not suitable for administration to a patient. Also, these solutions were found to lack long-term stability.

In U.S. Pat. No. 6,048,874, a method for making a pharmaceutical metolazone composition was described, comprising providing a solvent comprising a) propylene glycol, polyethylene glycol, polypropylene glycol, or glycerin; b) adding metolazone to the solvent, and c) heating the solvent and metolazone mixture. The method provided a metolazone concentration of 9 mg/mL, which is above the apparent equilibrium solubility. It is expected that these formulations would lack long-term stability.

In U.S. Pat. Nos. 7,923,447 and 9,427,398, dimethylacetamide is proposed as a solvent in parenteral metolazone formulations. This solvent is however considered to be a developmental toxicant (see OECD SIDS N,N-dimethylacetamide DMAC, 2001).

In U.S. Patent Publication No. 2018/0296556, non-aqueous, homogeneous metolazone solutions comprising a solubilized lipophilic agent and an amphiphilic liquid polymeric solvent were described. The formulations were essentially free of non-polymeric organic solvents, water, and non-solubilized particles, and the solubilized lipophilic pharmaceutical agent had a concentration of at least about 0.5 mg/mL, and further wherein the solution remains stable and essentially free of non-solubilized particles for at least 40 days when stored at room temperature of 25° C. However, this system too did not provide long-term stability as required in the pharmaceutical supply chain.

In view of the above, there remains a need in the art for metolazone formulations that can be used for parenteral administration and have adequate long-term storage stability for use in the pharmaceutical supply chain.

The objective of the present invention is to solve at least one or more of the problems as described above. In particular, the invention aims to provide a metolazone formulation with long-term storage stability.

### SUMMARY OF THE INVENTION

In an aspect, there are described novel metolazone lyophilizate compositions, comprising metolazone and a bulking agent.

In another aspect, there are described novel pre-lyophilizate compositions, comprising metolazone, a bulking agent, a solvent, and water.

In another aspect, there are described novel pharmaceutically acceptable diluents suitable to reconstitute the metolazone lyophilizate compositions.

In another aspect, there are described novel intravenous metolazone kits comprising the metolazone lyophilizate compositions and the pharmaceutically acceptable diluents.

In another aspect, there are described methods of reconstituting metolazone solutions suitable for parenteral administration.

In another aspect, there are described novel ready-to-use reconstituted metolazone solutions and uses of the reconstituted a metolazone solutions.

These and other aspects, which will become apparent during the following detailed description, have been achieved by the inventors' discovery of novel metolazone lyophilizate compositions.

### BRIEF DESCRIPTION OF THE DRAWINGS

FIG. 1 shows the tertiary butyl alcohol (TBA) content (mg/vial) based on the mannitol concentration (mg/mL).

FIG. 2 shows the level of impurity B (%) versus mannitol concentration (mg/mL).

FIG. 3 shows the level of impurity B (%) versus residual TBA concentration (mg/vial).

FIG. 4 shows metolazone solubility (mg/mL) versus TBA fraction (%) in the solvent.

### DETAILED DESCRIPTION OF THE INVENTION

Exemplary aspects of the invention are described herein. Although the following detailed description contains many specifics for purposes of illustration, a person of ordinary skill in the art will appreciate that variations and alterations to the following details are within the scope of the invention. Accordingly, the following aspects of the invention are set forth without any loss of generality to, and without imposing limitations upon, the claimed invention.

### Metolazone Lyophilizate Composition

An aspect of the invention involves a novel metolazone lyophilizate composition, comprising:
a. metolazone; and,
b. a bulking agent;
wherein the metolazone lyophilizate composition is in the form of a lyophilized powder.

In some aspects, the storage stability of the metolazone lyophilized powder is at least 3 months measured at 40° C. and/or 75% relative humidity. Storage stability is obtained if all known and unknown impurities are less than 0.5% w/w. An additional example is less than 0.2% w/w.

In some aspects, the metolazone lyophilized powder is reconstituted in a diluent to form a clear solution in less than about 5 minutes. Additional examples include less than about 4, 3 and/to 2 minutes.

In some aspects, about 2.5-20 mg of metolazone is present in the lyophilizate. Additional examples of the amount of metolazone present include about 2.5-10 mg. Further examples include about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, to/and about 20 mg.

In some aspects, the metolazone is micronized. Examples of micronized metolazone include a D90 less than 10 microns. D90 means that 90% of the sample has a particle diameter<10 microns. The method of measurement for particle size is a Malvern mastersizer.

In some aspects, the bulking agent is selected from mannitol, lactose, dextrose, sucrose and a cyclodextrin (e.g., hydroxypropyl-β-cyclodextrin). Examples of the amount of bulking agent present in the lyophilizate composition include about 2.5-1000 mg, about 25-500, about 25-150 mg, about 50-150 mg, about 2.5-60 mg, about 25-55 mg, and about 35-45 mg. Additional examples include about 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, to/and about 1000 mg.

In some aspects, the lyophilizate composition contains less than about 3500 ppm tertiary butyl alcohol. Additional examples include less than about 3000, 2500, to/and about 2000 ppm.

In some aspects, the amount of bulking agent (e.g., mannitol) present is sufficient to ensure the residual tertiary butyl alcohol (TBA) levels (amount of TBA remaining after lyophilization) is less than about 1.75 mg/vial. Additional examples of residual TBA remaining after lyophilization include less than about 1.6, 1.5, 1.4, 1.3, 1.2, 1.1, 1, 0.9, 0.8, 0.7, 0.6, 0.5, 0.4, 0.3 to/and about 0.2 mg/vial.

In another aspect, the lyophilizate composition, comprises:
a. about 2.5-20 mg metolazone; and,
b. about 2.5-1000 mg of a bulking agent is selected from mannitol, lactose, dextrose, sucrose and hydroxypropyl-β-cyclodextrin.

In another aspect, the lyophilizate composition, comprises:
a. about 2.5-10 mg metolazone; and,
b. about 2.5-500 mg of mannitol.

In another aspect, the lyophilizate composition, comprises:
a. about 2.5-10 mg metolazone; and,
b. about 2.5-50 mg of mannitol.

In another aspect, the lyophilizate composition, comprises:
a. about 2.5-10 mg metolazone; and,
b. about 5-15 mg of mannitol.

In another aspect, the lyophilizate composition, comprises:
a. about 2.5-10 mg metolazone; and,
b. about 2.5-10 mg of mannitol.

In another aspect, the lyophilizate composition, comprises:
a. about 5 mg metolazone; and,
b. about 25-55 mg of mannitol.

In another aspect, the lyophilizate composition, comprises:
a. about 5 mg metolazone; and,
b. about 50 mg of mannitol.

In another aspect, the lyophilizate composition, comprises:
a. about 5 mg metolazone; and,
b. about 100 mg of mannitol.

In another aspect, the lyophilizate composition, comprises:
a. about 5 mg metolazone; and,
b. about 150 mg of mannitol.

In another aspect, the lyophilizate composition, comprises:
a. about 10 mg metolazone; and,
b. about 50-150 mg of mannitol.

In another aspect, the lyophilizate composition, comprises:
a. about 10 mg metolazone; and,
b. about 50 mg of mannitol.

In another aspect, the lyophilizate composition, comprises:
a. about 10 mg metolazone; and,
b. about 100 mg of mannitol.

In another aspect, the lyophilizate composition, comprises:
a. about 10 mg metolazone; and,
b. about 150 mg of mannitol.

In another aspect, the invention involves a lyophilizate composition, wherein the composition is housed in a container.

Examples of containers include vials (e.g., clear or colored (e.g., amber))(e.g., glass, or plastic) and multi-chamber syringe presentations wherein the lyophilizate and the diluent are part of the same syringe (but in different chambers).

In another aspect, the invention involves a lyophilizate composition, wherein the container is an amber vial.

In another aspect, the invention involves a lyophilizate composition, wherein the container is a syringe.

### Pre-Lyophilizate Composition

In another aspect, the invention involves a liquid, metolazone pre-lyophilizate composition, comprising:
a. metolazone;
b. a bulking agent;
c. an alcohol; and,
d. water;
wherein the liquid, metolazone pre-lyophilizate composition forms a lyophilized powder after being subjected to lyophilization.

In some aspects, water (e.g., in the pre-lyophilizate compositions and in the diluents) is typically water suitable for injection (water for injection or WFI). In some aspects, if WFI is not used (e.g., in a diluent described herein), then typically the water will be treated (e.g., sterilized) in such a way as to make the water (or resulting composition) suitable for injection.

As described herein, the alcohol and water are removed during lyophilization to leave a lyophilizate or freeze-dried powder. The lyophilizate is stable (as described herein) and can be reconstituted in a diluent to provide a clear solution suitable for intravenous injection of metolazone.

In some aspects, the amount of metolazone present in the metolazone pre-lyophilization composition is about 0.1-2 mg/ml, about 0.1-1.5 mg/mL, and about 0.1-1 mg/mL. Additional examples include about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, to/and about 2 mg/mL.

In some aspects, the bulking agent is selected from mannitol, lactose, dextrose, sucrose and a cyclodextrin (e.g., hydroxypropyl-β-cyclodextrin). Examples of the amount of bulking agent present in the lyophilizate composition include about 2.5-500 mg, about 25-150 mg, about 50-150 mg, about 2.5-60 mg, about 25-55 mg, and about 35-45 mg. Additional examples include about 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 105, 110, 115, 120, 125, 130, 135, 140, 145, to/and about 150 mg.

In some aspects, the alcohol in the metolazone pre-lyophilization composition is tertiary butyl alcohol (TBA). Examples of the amount of TBA present include about 30-70% v/v (volume/volume or by volume), about 30-70% v/v, and about 40-60% v/v. Additional examples include about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 to/and about 70% v/v.

In another aspect, the invention involves a liquid, metolazone pre-lyophilizate composition, comprising:
a. metolazone;
b. a bulking agent selected from mannitol, lactose, dextrose, sucrose and hydroxypropyl-β-cyclodextrin;
c. tertiary butyl alcohol; and,
d. water.

In another aspect, the invention involves a liquid, metolazone pre-lyophilizate composition, comprising:
a. metolazone;
b. mannitol;
c. tertiary butyl alcohol; and,
d. water.

In another aspect, the pre-lyophilizate composition, comprises:
a. about 0.1-2 mg/ml metolazone;
b. about 1-50 mg/mL mannitol;
c. about 30-70% v/v tertiary butyl alcohol; and,
d. the remainder, water.

In another aspect, the pre-lyophilizate composition, comprises:
a. about 0.1-1.5 mg/ml metolazone;
b. about 2.5-60 mg/mL mannitol;
c. about 30-70% by volume tertiary butyl alcohol; and,
d. the remainder, water.

In another aspect, the pre-lyophilizate composition, comprises:
a. about 0.1-1 mg/ml metolazone;
b. about 5-10 mg/mL mannitol;
c. about 40-60% by volume tertiary butyl alcohol; and,
d. the remainder, water.

In another aspect, the pre-lyophilizate composition, comprises:
a. about 0.1-1 mg/ml metolazone;
b. about 5-10 mg/mL mannitol;
c. about 50% by volume tertiary butyl alcohol; and,
d. the remainder, water.

In another aspect, the pre-lyophilizate composition, comprises:
a. about 1 mg/ml metolazone;
b. about 5 mg/mL mannitol;
c. about 50% by volume tertiary butyl alcohol; and,
d. the remainder, water.

In another aspect, the pre-lyophilizate composition, comprises:
a. about 1 mg/ml metolazone;
b. about 10 mg/mL mannitol;
c. about 50% by volume tertiary butyl alcohol; and,
d. the remainder, water.

In another aspect, the pre-lyophilizate composition, comprises:
a. about 0.5 mg/ml metolazone;
b. about 5 mg/mL mannitol;
c. about 50% by volume tertiary butyl alcohol; and,
d. the remainder, water.

In another aspect, the pre-lyophilizate composition, comprises:
a. about 0.5 mg/ml metolazone;
b. about 10 mg/mL mannitol;
c. about 50% by volume tertiary butyl alcohol; and,
d. the remainder, water.

In some aspects, the tertiary butyl alcohol (TBA) to water ratio (volume:volume) is at most about 60% TBA and at least about 40% water. Additional examples of the TBA:water ratio include 50:50 and 35:65.

In another aspect, the pre-lyophilizate composition, further comprises:
a non-ionic solubilizer.

In some aspects, the non-ionic solubilizer is macrogol-15-hydroxystearate.

Macrogol-15-hydroxystearate is also known as polyethylene glycol 660 12-hydroxystearate and polyoxyethylated 12-hydroxystearic acid and under the trade name Kolliphor® HS 15.

Examples of the amount of macrogol-15-hydroxystearate present in the pre-lyophilizate composition include about 1-15 mg/mL about 1-10 mg/mL, and about 1-4 mg/mL. Additional examples include about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 to/and about 15 mg/mL.

In another aspect, the pre-lyophilizate composition, further comprises:
about 1-15 mg/mL macrogol-15-hydroxystearate.

In some aspects, the invention involves a method of preparing a metolazone lyophilizate composition, comprising the step of lyophilizing a metolazone pre-lyophilizate composition described herein to form a lyophilizate composition described here.

In some aspects, dimethylacetamide (DMAC) is absent from the metolazone pre-lyophilizate composition, the metolazone lyophilizate composition, and/or the final drug product (after reconstitution of the metolazone lyophilizate composition).

### Pharmaceutically Acceptable Diluent

In another aspect, the invention involves a pharmaceutically acceptable diluent, comprising:
a. a solubilizer;
b. a buffer; and,
c. water;
wherein the pH of the diluent is about 6-8.

In some aspects, the diluent is prepared by first preparing a buffer (e.g., TRIS buffer or phosphate buffer) and adjusting the pH (e.g., addition of acid or base) to the desired pH (e.g., pH 7.5). The solubilizer is then mixed into the buffer.

In some aspects, the diluent, once formed, is filtered and sterilized. For example, the diluent can be aseptically filtered through 0.2 micron filters and filled into a container (e.g., a glass vial). The container can then be sterilized at 121° C. for 30 mins.

In some aspects, the pH of the diluent is about 6, 6.5, 7, 7.5 to/and 8.0.

In some aspects, the solubilizer is polyethylene glycol 400 (PEG-400). Examples of the amount of PEG-400 include about 5-70% w/v (weight to volume), about 10-50% w/v, about 25-45% w/v, and about 30-40% w/v. Additional examples of the amount of polyethylene glycol 400 include about 5, 10, 15, 120, 25, 30, 35, 40, 45, 50, 55, 60, 65, to/and 70% w/v.

In some aspects, the buffer is TRIS (tris(hydroxymethyl)aminomethane) or phosphate buffer. Examples of the amount of buffer include about 0.1-100 mM (millimolar). Additional examples of the amount of buffer present include about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, to/and 100 mM.

In another aspect, the diluent, comprises:
a. about 10-40% w/v polyethylene glycol 400;
b. TRIS buffer or phosphate buffer; and,
c. the remainder, water;
wherein the pH of the diluent is about 7-8.

In another aspect, the diluent, comprises:
a. about 25-45% w/v polyethylene glycol 400;
b. TRIS buffer or phosphate buffer; and,
c. the remainder, water;
wherein the pH of the diluent is about 7-8.

In another aspect, the diluent, comprises:
a. about 30-40% w/v polyethylene glycol 400;
b. TRIS buffer or phosphate buffer; and,
c. the remainder, water;
wherein the pH of the diluent is about 7.5.

In another aspect, the diluent, comprises:
a. about 40% w/v polyethylene glycol 400;
b. TRIS buffer; and,
c. the remainder, water;
wherein the pH of the diluent is about 7.5.

In another aspect, the diluent, comprises:
a. 40% w/v polyethylene glycol 400;
b. phosphate buffer; and,
c. the remainder, water;
wherein the pH of the diluent is about 7.5.

In some aspects, the solubilizer to drug weight ratio (weight:weight) is from about 25-2000. Additional examples include a PEG-400:metolazone weight ratio of from about 25-2000. These weight ratios can be before reconstitution (e.g., the ratio is for the solubilized (e.g. PEG-400) in the diluent and metolazone in the lyophilizate composition) of after reconstitution.

In another aspect, the diluent, further comprises:
a non-ionic solubilizer.

In some aspects, the non-ionic solubilizer in the diluent is macrogol-15-hydroxystearate. Examples of the amount of macrogol-15-hydroxystearate present in the diluent include about 1-5% w/v (weight to volume) and about 2-4% w/v. Additional examples include about 1, 1.5, 2, 2.5, 3, 3.5, to/and about 4% w/v.

In another aspect, the diluent, further comprises: about 1-5% w/v macrogol-15-hydroxystearate.

In another aspect, the diluent, further comprises: about 2-4% w/v macrogol-15-hydroxystearate.

### Intravenous Metolazone Kit

In another aspect, the invention involves an intravenous metolazone kit, comprising:
a. a first container, comprising: a metolazone lyophilizate composition described herein; and,
b. a second container, comprising: pharmaceutically acceptable diluent described herein.
wherein the diluent is suitable to reconstitute the lyophilizate composition into a metolazone solution having a pH about 5.0-8.0 being suitable for parenteral administration.

In another aspect, the kit further comprises a connector for holding the first and second containers. This connector acts as an aid in the reconstitution of the IV product. In some aspects, the connector is permanently affixed to the first and second containers (e.g., the containers are directly attached to one another or attached via a spacer). In some aspects, the connector can be attached to the containers (e.g., the containers are placed into a holder) when it is time to reconstitute the product.

In another aspect, the invention involves a metolazone lyophilizate kit, comprising:
a. a first container, comprising: a metolazone lyophilizate composition in the form of a lyophilized powder, comprising:
   i. metolazone; and,
   ii. a bulking agent; and,
b. a second container, comprising: pharmaceutically acceptable diluent, comprising:
   i. a solubilizer;
   ii. a buffer; and,
   iii. water;
wherein the diluent is suitable to reconstitute the lyophilizate composition into a drug product having a pH about 5.0-8.0 for parenteral administration.

In some aspects, examples of the pH reconstituted drug product include about 5.0, 5.5, 6, 6.5, 7, 7.5 to/and 8.0.

In another aspect, the diluent, further comprises: a non-ionic solubilizer.

In some aspects, the non-ionic solubilizer in the diluent is macrogol-15-hydroxystearate. Examples of the amount of macrogol-15-hydroxystearate present in the diluent include about 1-5% w/v (weight to volume) and about 2-4% w/v. Additional examples include about 1, 1.5, 2, 2.5, 3, 3.5, to/and about 4% w/v.

In another aspect, the diluent, further comprises:
about 1-5% w/v macrogol-15-hydroxystearate.

In another aspect, the diluent, further comprises:
about 2-4% w/v macrogol-15-hydroxystearate.

In some aspects, the metolazone lyophilizate composition contains less than about 10,000 ppm, 5000 ppm, 3500 ppm tertiary butyl alcohol. Additional examples include less than about 3000, 2500, to/and about 2000 ppm.

In another aspect, in the metolazone lyophilizate kit:
the metolazone lyophilizate composition, comprises:
   i. about 2.5-20 mg metolazone; and,
   ii. about 2.5-1000 mg of mannitol; and,
the diluent, comprises:
   i. about 25-45% w/v polyethylene glycol 400;
   ii. TRIS buffer or phosphate buffer; and,
   iii. the remainder, water;
wherein the pH of the diluent is about 7-8.

In another aspect, in the metolazone lyophilizate kit:
the metolazone lyophilizate composition, comprises:
   i. about 2.5-10 mg metolazone; and,
   ii. about 25-500 mg of mannitol;
the diluent, comprises:
   i. about 30-40% w/v polyethylene glycol 400;
   ii. TRIS buffer or phosphate buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 50-150 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 50 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 100 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 150 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 50-150 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 50 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 100 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 150 mg of mannitol;
the diluent, comprises:
   i. about 40% w/v polyethylene glycol 400;
   ii. TRIS buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 50-150 mg of mannitol;
the diluent, comprises:
   i. about 40% w/v polyethylene glycol 400;
   ii. phosphate buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 50 mg of mannitol;
the diluent, comprises:
   i. about 40% w/v polyethylene glycol 400;
   ii. phosphate buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 100 mg of mannitol;
the diluent, comprises:
   i. about 40% w/v polyethylene glycol 400;
   ii. phosphate buffer; and,
   iii. the remainder, water;
wherein the pH of the diluent is about 7.5.

In another aspect, in the metolazone lyophilizate kit:
the metolazone lyophilizate composition, comprises:
   i. about 5 mg metolazone; and,
   ii. about 150 mg of mannitol;
the diluent, comprises:
   i. about 40% w/v polyethylene glycol 400;
   ii. phosphate buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 50-150 mg of mannitol;
the diluent, comprises:
   i. about 40% w/v polyethylene glycol 400;
   ii. phosphate buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 50 mg of mannitol;
the diluent, comprises:
   i. about 40% w/v polyethylene glycol 400;
   ii. phosphate buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 100 mg of mannitol;
the diluent, comprises:
   i. about 40% w/v polyethylene glycol 400;
   ii. phosphate buffer; and,
   iii. the remainder, water;
wherein the pH of the diluent is about 7.5.

In another aspect, in the metolazone lyophilizate kit:
the metolazone lyophilizate composition, comprises:
   i. about 10 mg metolazone; and,
   ii. about 150 mg of mannitol;
the diluent, comprises:
   i. about 40% w/v polyethylene glycol 400;
   ii. phosphate buffer; and,
   iii. the remainder, water;
wherein the pH of the diluent is about 7.5.

In some aspects, the volume of diluent is about 5-20 mL, about 5-10 mL, and about 10-20 mL. Additional examples of the volume of diluent include about 1, 2, 3, 4, 5, 6, 7, 8, 9, 10, 11, 12, 13, 14, to/and about 15 mL.

In some aspects, the ratio of diluent to metolazone (volume:weight) in the kit is from about 1-20.

### Reconstituting a Metolazone Solution

In another aspect, the invention involves a process for reconstituting a metolazone lyophilizate composition, the process comprising:
contacting a metolazone lyophilizate composition described herein with a pharmaceutically acceptable diluent described herein; and,
mixing the resulting solution until clear;
wherein the resulting clear solution is suitable for parenteral administration.

In another aspect, in the reconstituting process, a clear solution is obtained in less than about 5 minutes. Clear refers to no particulate matter being visible to the naked eye. Additional examples of the time to obtain a clear solution include less than about 4, 3 and/to 2 minutes.

In some aspects, the ratio of diluent to metolazone (volume:weight) in the reconstituted metolazone solution is from about 1-20.

### Ready-to-Use and Use of a Reconstituted Metolazone Solution

In another aspect, the invention involves a ready-to-use injectable metolazone formulation for parenteral administration, obtained by mixing the metolazone lyophilizate composition described herein with a pharmaceutically acceptable diluent described herein, wherein the pH of the ready-to-use injectable metolazone formulation obtained is about 5-8.

For example, the ready-to-use injectable metolazone formulation for parenteral administration, is obtained by mixing
a metolazone lyophilizate composition, comprising:
   i. about 2.5-20 mg metolazone; and,
   ii. about 2.5-1000 mg of mannitol; and,
with about 5-15 mL of a diluent, comprising
   i. about 25-45% w/v polyethylene glycol 400;
   ii. TRIS buffer or phosphate buffer; and,
   iii. the remainder, water;
wherein the pH of the diluent is about 7-8.

In another example, the diluent in the ready-to-use formulation, further comprises: about 2-4% w/v macrogol-15-hydroxystearate.

In another aspect, the invention involves a method of treating a patient in need of a natriuretic and/or diuretic treatment, comprising:
contacting a metolazone lyophilizate composition described herein with a pharmaceutically acceptable diluent described herein; and,
mixing the resulting solution until a clear, reconstituted metolazone solution is obtained;
administering to the patient in need of the natriuretic and/or diuretic treatment a therapeutically effective amount of the reconstituted metolazone.

In another aspect, the therapeutically effective amount of the reconstituted metolazone comprises about 2.5-20 mg metolazone. Additional examples of the therapeutically effective amount of metolazone include about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, to/and about 20 mg.

In another aspect, the patient in need of a natriuretic and/or diuretic treatment is suffering from congestive heart failure or a renal disease.

In another aspect, the patient is suffering from loop diuretic resistance.

In another aspect, the patient is suffering from furosemide resistance.

In another aspect, the patient is suffering from hypertension.

The invention may be embodied in other specific forms without departing from the spirit or essential attributes thereof. This invention encompasses all combinations of aspects of the invention noted herein. It is understood that any and all embodiments of the invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

### Definitions

The examples provided in the definitions present in this application are non-inclusive unless otherwise stated. They include but are not limited to the recited examples.

When introducing elements of the present disclosure or an aspect thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

About includes +/−10% of the numerical value.

“Subject” or “patient” are used interchangeably and mean all members of the animal kingdom (e.g., humans).

“Mammal” and “patient” cover warm blooded mammals that are typically under medical care (e.g., humans and domesticated animals). Examples include feline, canine, equine, bovine, non-human primate, and human, as well as just human.

Lyophilizate is a freeze-dried product (a product made lyophilization).

Lyophilization or freeze-drying is a low temperature dehydration process that involves freezing a solution, lowering the pressure, and removing the ice by sublimation.

Other features of the invention will become apparent during the following descriptions of exemplary embodiments that are given for illustration of the invention and are not intended to be limiting thereof.

### EXAMPLES

Kolliphor HS 15 is a yellowish white paste at room temperature of 25° C. that becomes a liquid at approximately 30° C. It dissolves in water, ethanol, and 2-propanol to form clear solutions. Its solubility in water decreases with increasing temperatures. It is insoluble in liquid paraffin. The compendial name is Polyethylene glycol 660 12-hydoxystearate or Macrogol-15-hydroxystearate

Lyophilization is a process which can lead to a product with extremely small particle size and can facilitate rapid dissolution of metolazone. Hence a 2 vial/container system is hypothesized, wherein one container will have Lyophilized Metolazone, and the second container will have aseptically filtered and terminally sterilized diluent.

### The API Polymorph

Metolazone USP is obtained from a quality source. Metolazone (API) is reported to have polymorphism. The Form I metolazone was consistently produced and supplied by the supplier. The form of API may impact solubility of the API. In the context of the formulations described herein, the form of API may not make a significant difference as ultimately the API is dissolved in a suitable agent before lyophilization.

### Particle Size Distribution [PSD] of the API

The rate of dissolution will depend upon the PSD of the API; hence it was observed that the smaller the PSD, the faster the dissolution rate. Thus, it is beneficial to have a micronized API. For example, a D90 less than 10 microns can be used (90% of the sample has a particle diameter<10 μm), measured by a Malvern Mastersizer.

Before initiating solvent based Lyophilization trials, a detail assessment of metolazone solubility in Water+TBA (Tertiary butyl alcohol) was carried out to identify optimum ratio of TBA:Water.

TABLE 1
Tertiary butyl alcohol-water solubility trials

| Trials | 1 | 2 | 3 | 4 | 5 | 6 | 7 |
|---|---|---|---|---|---|---|---|
| Composition | WFI | 20% | 30% | 40% | 50% | 60% | 30% |
| | | TBA | TBA | TBA | TBA | TBA | TBA |
| Metolazone (mg) | 5.04 | 10.08 | 15.12 | 30.24 | 30.24 | 30.24 | 20.16 |
| TBA (g) | 0 | 3.1 | 4.65 | 6.2 | 7.75 | 9.3 | 4.65 |
| Water for | Q.S. to | Q.S. to | Q.S. to | Q.S. to | Q.S. to | Q.S. to | Q.S. to |
| Injection | 10 mL | 20 mL | 20 mL | 20 mL | 20 mL | 20 mL | 20 mL |
| Observations | Insoluble | opalescent | clear | clear | clear | clear | clear |

Based on these trials it appears that TBA:Water in a ratio of at least 30:70 will provide a clear solution of metolazone.

### Example 1: Composition: #01

| Ingredients | Concentration (mg/mL) | Batch quantity (gm) |
|---|---|---|
| Metolazone | 1.00 | 0.3 |
| HPBCD [20% w/w] | 216 | 64.80 |
| Mannitol 2.5% | 25 | 7.5 |
| WFI | Q.S to 1.0 mL | Q.S to 300.0 ml |

Primary packing material

| | |
|---|---|
| Clear Glass vial | 30 mL tubular glass vial |
| Rubber stopper | 20 mm uncoated rubber stopper |

\hat{ }Target strength 10 mg/vial

The following procedure was used for Example 1:

| | |
|---|---|
| a | dissolved HPBCD (hydroxypropyl-β-cyclodextrin (bulking agent)) |
| | in WFI under constant stirring, |
| b | added metolazone in the vortex to form a clear solution, |
| c | added bulking agent mannitol |
| d | filtered the solution through 0.22 microns filter |
| e | filled the solution in USP Type I clear glass vials, and |
| f | lyophilized the solution as per below parameters. |

### HPBCD is.

Lyophilization Parameters

| | Temp. | Ramp | Hold | Vacuum |
|---|---|---|---|---|
| Stages | (° C.) | (min) | (min) | (mTorr) |
| Pre-freeze | 5 | — | — | — |
| Thermal Treatment | 5 | 45 | 90 | — |
| (Freezing) | −5 | 40 | 120 | — |
| | −35 | 120 | 200 | — |
| | −15 | 80 | 200 | — |
| | −35 | 80 | 200 | — |
| Evacuation (shelf driven) | −35 | — | — | 150 |
| Primary drying | −35 | 10 | 30 | 150 |
| | −30 | 40 | 500 | 150 |
| | −15 | 160 | 3400 | 150 |
| Secondary drying | 5 | 120 | 90 | 150 |
| | 20 | 60 | 150 | 150 |

A nice cake was formed which can be reconstituted with suitable diluent such as water for injection with suitable pH 5.5 to 8. It was determined that the diluent may optionally contain surfactants to provide a clear solution.

### Example 2: Composition #02

| Ingredients | Concentration (mg/mL) | Batch quantity requirement | |
|---|---|---|---|
| Metolazone | 0.5 | 150 | mg |
| TBA | 232.5* | 69.75 | g* |
| Mannitol | 50 | 15 | g |

| | | |
|---|---|---|
| WFI | Q.S to 1.0 mL | Q.S to 300.0 mL |
| (water for injection) | | |

Primary packing material

| | |
|---|---|
| Glass vial | 50 mL tubular glass vial |
| Rubber stopper | 20 mm uncoated rubber stopper |

*TBA density 0.775 g/mL used for calculation. Total TBA is 30% v/v.

Detailed manufacturing procedure:
Step-1: Transfer required quantity of drug substance to suitable SS316 vessel.
Step-2: Add the required quantity of solvent (TBA) to step 1 and mix using an overhead stirrer at 500-800 rpm until a clear solution is observed. In the case of TBA if no clear solution is observed, add 35% of batch quantity of WFI and mix till clear solution observed.
Step-3: Once clear solution obtained, add WFI up to 90% of batch quantity and mix using an overhead stirrer at 500-800 rpm to get a clear solution.
Step-4: Add batch quantity of mannitol and mix using overhead stirrer at 500-800 rpm to get a clear solution. Once a clear solution is obtained, make up the final volume with WFI.
Step-5: Filter the solution from step 4 using 0.224 PVDF filter using SS pressure vessel with Nitrogen. Perform a nitrogen purging in filtered bulk solution for 1 hr.
Step-6: Fill the required volume of filtered solution in a 50 mL or suitable glass vial and half stopper with single vent uncoated rubber stopper.
Step-7: Load the filled vial to lyophilizer and initiate the lyophilization cycle as per provided parameters.
Step-8: Once lyophilization cycle is over, release the vacuum partially with nitrogen and stopper the vial.
Step-9: After unloading, seal the vials using aluminium seal with plastic flip off. Store the vial in carton box at room temperature of 25° C. for future experiments.

Lyophilization Parameters

| | Temp. | Ramp | Hold | Vacuum |
|---|---|---|---|---|
| Stages | (° C.) | (min) | (min) | (mTorr) |
| Pre-freeze | 5 | — | — | — |
| Thermal Treatment | 5 | 45 | 90 | — |
| (Freezing) | −5 | 40 | 120 | — |
| | −35 | 120 | 200 | — |
| | −15 | 80 | 200 | — |
| | −35 | 80 | 200 | — |
| Evacuation (shelf driven) | −35 | — | — | 150 |
| Primary drying | −35 | 10 | 30 | 150 |
| | −30 | 40 | 500 | 150 |
| | −15 | 160 | 3400 | 150 |
| Secondary drying | 5 | 120 | 90 | 150 |
| | 20 | 60 | 150 | 150 |

No collapse was observed in any of the lyophilized vial. White color compact cake or powder was observed. No vial breakage was observed at the end of Lyophilization cycle. Lyophilized cake was reconstituted with suitable diluent containing PEG 400 at 30% w/v in phosphate buffer pH 7.5. The reconstitution happened in less than 3 mins.

### Example 3: Composition #06 (Reduction of Lyophilization Time from 92 to 72 Hrs)

| Ingredients | Concentration (mg/mL) | Batch quantity requirement | |
|---|---|---|---|
| Metolazone | 0.5 | 150 | mg |
| TBA | 232.5* | 69.75 | g* |
| Mannitol | 50 | 15 | g |

| | | |
|---|---|---|
| WFI | Q.S to 1.0 mL | Q.S to 300.0 mL |

Primary packing material

| | |
|---|---|
| Glass vial | 50 mL tubular glass vial |
| Rubber stopper | 20 mm uncoated rubber stopper |

*TBA density 0.775 g/mL used for calculation. Total TBA is 30% v/v.

Lyophilization Parameters

| | Temp. | Ramp | Hold | Vacuum |
|---|---|---|---|---|
| Stages | (° C.) | (min) | (min) | (mTorr) |
| Pre-freeze | 5 | — | — | — |
| Thermal Treatment | 5 | 45 | 90 | — |
| (Freezing) | −5 | 40 | 120 | — |
| | −35 | 120 | 200 | — |
| | −15 | 80 | 200 | |
| | −35 | 80 | 200 | |
| Evacuation (shelf driven) | −35 | — | — | 150 |
| Primary drying | −35 | 10 | 30 | 150 |
| | −30 | 40 | 500 | 150 |
| | −15 | 160 | 1885 | 150 |
| Secondary drying | 5 | 70 | 90 | 150 |
| | 20 | 60 | 240 | 150 |

Total cycle time was reduced to approx. 72 hours from 96 hours of previous trial. Observations were like the composition trial 2. No collapse was observed in any of the lyophilized vial. White color compact cake or powder was observed. No vial breakage was observed at the end of Lyophilization cycle.

Reconstitution with 30% PEG 400 and 70% phosphate buffer (pH 7.4): Reconstitution vehicle was prepared by mixing 30% PEG 400 in phosphate buffer (pH 7.4). 10 mL of reconstitution vehicle was added into the lyophilized product vial using 10 mL plastic syringe. Vial was swirled for approximately 1 minute. No clear solution observed. Another 10 mL of reconstitution vehicle was added to vial using 10 mL plastic syringe and vial was swirled for approximately 1-2 minute. Clear solution observed. Vial was kept at room temperature for 24 hours for observation. Clear solution without any precipitation was observed. It was observed that 15-20 mL of diluent provided a faster reconstitution. Since less than 15 mL of diluent would be beneficial, further optimization of diluent was carried out.

Diluent is used for reconstitution of the lyophilized cake in 2 vial kit and can be prepared by dissolving a buffer in water and adjusting a pH between 5-8. To this solution can be added a suitable surfactant or solubilizer to obtain a diluent system that can then be filtered (e.g., through 0.22-micron filters) and then filled into a container (e.g., a vial) to carry out terminal sterilization, if required.

Table 2 exemplifies some of the studies conducted on diluent with different solubilizers and co-solvents

TABLE 2
Diluent optimization studies conducted on Composition #06
Reconstitution study

| No of | | | |
|---|---|---|---|
| Trials | Trial Details | Remarks | Observations |
| Trial- | Reconstitution | Included HS 15 of 4% | Clear colorless solution. |
| 1 | with 40% PEG- | additionally and the | Reconstitution Time: <120 seconds |
| | 400 + Kolliphor | concentration of PEG- | Solution was found to be clear without |
| | HS 15 4% in 15 | 400 increased to 40% | any particles, slight foam was observed |
| | mL of Water for | and WFI mixture. | on reconstitution which got cleared |
| | Injection mixture | | after some time. |
| Trial- | Reconstitution | PEG-400 concentration | Clear Colorless solution. |
| 2 | with 30% PEG- | kept constant and | Reconstitution Time: <120 seconds |
| | 400 + Kolliphor | Included HS 15 of 4% | Solution was found to be clear without |
| | HS 15 4% in 15 | additionally and WFI | any particles, slight foam was observed |
| | mL of Water for | mixture. | on reconstitution which cleared after |
| | Injection mixture | | some time. |
| Trial- | Reconstitution | Included ethanol 40% | Clear colorless solution. |
| 3 | with 40% Ethanol | instead of the 30% PEG- | Reconstitution Time: <120 seconds |
| | in 15 mL of Water | 400 and WFI mixture. | Solution was found to be clear without |
| | for Injection | | any particles. |
| | mixture | | |
| Trial- | Reconstitution | Included Ethanol of 20% | Clear colorless solution. |
| 4 | with 30% PEG- | additionally to the 30% | Reconstitution Time: <120 seconds |
| | 400 + Ethanol | PEG-400 and WFI | Solution was found to be clear without |
| | 20% in 15 mL of | mixture. | any particles. |
| | Water for | | |
| | Injection mixture | | |
| Trial- | Reconstitution | Will check the | Clear colorless solution. |
| 5 | with 30% PEG- | reconstitution with | Reconstitution Time: <180 seconds |
| | 400 in 15 mL | freshly prepared diluent | Solution was found to be clear without |
| | Phosphate buffer | as per the previous | any particles. |
| | of pH 7.5 | method. | |
| Trial- | Reconstitution | Included HS 15 of 4% | Clear colorless solution. |
| 6 | with 40% PEG- | additionally and the | Reconstitution Time: <120 seconds |
| | 400 + Kolliphor | concentration of PEG- | Solution was found to be clear without |
| | HS 15 4% in 15 | 400 increased to 40% | any particles, slight foam was observed |
| | mL of Phosphate | and Phosphate buffer pH | on reconstitution which got cleared |
| | buffer pH 7.5 | 7.5 mixture. | after some time. |
| | mixture | | |
| Trial- | Reconstitution | PEG-400 concentration | Clear colorless solution. |
| 7 | with 30% PEG- | kept constant and | Reconstitution Time: <120 seconds |
| | 400 + Kolliphor | Included HS 15 of 4% | Solution was found to be clear without |
| | HS 15 4% in 15 | additionally and | any particles, slight foam was observed |
| | mL of Phosphate | Phosphate buffer pH 7.5 | on reconstitution which got cleared |
| | buffer pH 7.5 | mixture. | after some time. |
| | mixture | | |
| Trial- | Reconstitution | Included ethanol 40% | Clear colorless solution. |
| 8 | with 40% Ethanol | instead of the 30% PEG- | Reconstitution Time: <120 seconds |
| | in 15 mL of | 400 and Phosphate | Solution was found to be clear without |
| | Phosphate buffer | buffer pH 7.5 mixture. | any particles. |
| | pH 7.5 mixture | | |
| Trial- | Reconstitutions | Included Ethanol of 20% | Clear Colorless solution. |
| 9 | with 30% PEG- | additionally to the 30% | Reconstitution Time: <120 seconds |
| | 400 + Ethanol | PEG-400 and Phosphate | Solution was found to be clear without |
| | 20% in 15 mL of | buffer pH 7.5 mixtures. | any particles. |
| | Phosphate buffer | | |
| | pH 7.5 mixture | | |

Reconstituted vials were kept on hold for observation at room temperature for 24 hours and after 24 hours all the reconstituted vials were found to be clear Colorless with no signs of precipitation.

Based on these trials it was evident that PEG, Kolliphor HS 15, and ethanol can become a part of effective diluent system in 2-vial kit.

### Example 4: Composition #07

| Ingredients | Concentration (mg/mL) | Batch quantity requirement | |
|---|---|---|---|
| Metolazone | 0.5 | 503.97 | mg |
| TBA | 232.5* | 232.500 | g* |
| Mannitol | 50 | 50 | g |

| | | |
|---|---|---|
| WFI | Q.S to 1.0 mL | Q.S to 1000.0 mL |

Primary packing material

| | |
|---|---|
| Glass vial | 50 mL tubular glass vial |
| Rubber stopper | 20 mm uncoated rubber stopper |

*TBA density 0.775 g/mL used for calculation. Total TBA is 30% v/v.

### Observations:

Based on the results of Example 3, the same lyophilization parameters were used for Example 4. The total cycle time was approx. 72 hours. Observations were like the #006 trial. No collapse was observed in any of the lyophilized vial. White color compact cake was observed in few vials and in few vials fluffy powder was observed.

The samples of the batch were studied for stability at accelerated condition at 40° C./75% RH (relative humidity), intermediate condition at 30° C./65% RH, and room temperature 25° C./60% RH. Upon withdrawal the samples were tested for its assay and impurities by HPLC method.

Upon withdrawal the samples were tested for its assay and impurities by HPLC, using a modified USP method (USP29-NF24) with the following modifications: GL Science, Inertsil ODS 3V, 150×4.6 mm, 5 μm as column, ACN:MeOH:Buffer (10:30:60) v/v as mobile phase, MeOH:H₂O (50:50) v/v as diluent, 10 μL as injection volume, and 0.1 mg/mL of metolazone sample and reference solution. The observations of the batch are recorded in the below table.

Stability of Composition #07

| Storage | | RT | 25° C./60% RH | | |
|---|---|---|---|---|---|
| condition | | Initial | 2 M | 3 M | 6 M |
| Name | RRT | % Impurity | | | |
| USP Impurity A-Desmethyl | 0.71 | 0.31 | 0.31 | 0.30 | 0.30 |
| Metolazone | | | | | |
| USP Impurity B-Metolazone | 1.15 | <0.05 | 0.08 | 0.08 | 0.09 |
| benzamide analog (DP2) | | | | | |
| USP Impurity C-meta- | 1.31 | 0.07 | 0.08 | 0.08 | 0.07 |
| Metolazone | | | | | |
| Impurity D | 1.36 | | ND | ND | ND |
| USP Impurity E-Di-dehydro | 1.45 | 0.13 | 0.08 | 0.11 | 0.12 |
| Metolazone (DP4) | | | | | |
| Unspecified UKI | 2.01 | | <0.05 | <0.05 | ND |
| Total Impurities | | 0.51 | 0.55 | 0.57 | 0.58 |
| ASSAY | | 96.0 | 95.6 | 94.2 | 96.3 |

Stability of Composition #07-continued

| | Storage | | | | | | |
|---|---|---|---|---|---|---|---|
| | 30° C./65% RH | | | 40° C./75% RH | | | |
| | condition | | | | | | |
| | 2 M | 3 M | 6 M | 1 M | 2 M | 3 M | 6 M |
| Name | % Impurity | | | | | | |
| USP Impurity | 0.32 | 0.35 | 0.32 | 0.31 | 0.34 | 0.37 | 0.32 |
| A-Desmethyl | | | | | | | |
| Metolazone | | | | | | | |
| USP Impurity | 0.12 | 0.15 | 0.09 | 0.07 | 0.20 | 0.12 | 0.14 |
| B-Metolazone | | | | | | | |
| benzamide | | | | | | | |
| analog (DP2) | | | | | | | |
| USP Impurity C-meta- | 0.07 | 0.08 | 0.07 | 0.07 | 0.07 | 0.07 | 0.06 |
| Metolazone | | | | | | | |
| Impurity D | ND | ND | ND | ND | ND | ND | ND |
| USP Impurity | 0.11 | 0.09 | 0.10 | 0.08 | 0.10 | 0.14 | 0.10 |
| E-Di-dehydro | | | | | | | |
| Metolazone (DP4) | | | | | | | |
| Unspecified UKI | <0.05 | <0.05 | ND | <0.05 | <0.05 | <0.05 | ND |
| Total Impurities | 0.62 | 0.67 | 0.58 | 0.53 | 0.71 | 0.70 | 0.62 |
| ASSAY | 93.8 | 98.2 | 95.5 | 96.5 | 95.7 | 97.3 | 95.9 |

ND: Not detected

Lyophilized metolazone showed excellent stability in all conditions thus providing at least a 24M (month) shelf life based on satisfactory accelerated stability conditions.

### Example 5: Composition: #10

| Ingredients | mg/mL | | mg/vial | |
|---|---|---|---|---|
| Metolazone | 1 | mg | 10 | mg |
| Tertiary butyl alcohol | 348.7 | mg | 3487 | mg |
| (TBA-45.0%) $* | | | | |
| Mannitol | 50.0 | mg | 500 | mg |

| | | | |
|---|---|---|---|
| Water for injection* | Q.s to mL | Q.s to 10 | mL |

*Removed during lyophilization.
$: TBA density 0.775 g/mL used for calculation

The manufacturing process was kept like earlier batches, reconstitution diluent or batches were prepared and filtered through 0.2-micron filters and were either exposed or not exposed to autoclave to check performance of different diluent prepared in Phosphate buffer or TRIS buffer. The following table provides the diluent assessment.

Composition #10
10 mL Diluent assessment

| | | | Diluent |
|---|---|---|---|
| Batch No. | Batch Details | Autoclaved? | pH |
| 013A | Diluent prepared with | Sterile Filtered | 7.888 |
| | 30% PEG-400 + 2% Kolliphor | Unautoclaved | |
| 013AA | HS 15 & Phosphate buffer | Autoclaved at | 8.045 |
| | Q.S to 100% | 121° C. for | |
| | | 15 minutes | |
| 013B | Diluent prepared with | Sterile Filtered | 8.237 |
| | 40% PEG-400 & Phosphate | Unautoclaved | |
| 013BA | buffer Q.S to 100% | Autoclaved at | 8.19 |
| | | 121° C. for | |
| | | 15 minutes | |
| 014A | Diluent prepared with | Sterile Filtered | 7.523 |
| | 30% PEG-400 + 2% Kolliphor | Unautoclaved | |
| 014AA | HS 15 & TRIS buffer Q.S to | Autoclaved at | 7.532 |
| | 100% | 121° C. for | |
| | | 15 minutes | |
| 014B | Diluent prepared with | Sterile Filtered | 7.567 |
| | 40% PEG-400 & TRIS buffer | Unautoclaved | |
| 014BA | Q.S to 100% | Autoclaved at | 7.548 |
| | | 121° C. for | |
| | | 15 minutes | |

Composition #10: 10 mL Diluent assessment-continued

| | Reconstitution | | pH |
|---|---|---|---|
| Batch No. | Time (s) | observation | observed |
| 013A | 120-180 | clear solution | 7.691 |
| 013AA | 120-180 | | 7.73 |
| 013B | 120 | clear solution | 7.894 |
| 013BA | 120 | | 7.884 |
| 014A | 120-180 | clear solution | 7.65 |
| 014AA | 120-180 | | 7.604 |
| 014B | 120 | clear solution | 7.631 |
| 014BA | 120 | clear solution | 7.633 |

It was observed that in most of the cases a clear solution of metolazone was obtained after reconstitution with diluent containing PEG or Kolliphor HS 15 alone or in combination.

It is noted that the composition with 50 mg/mL mannitol leads to almost 500 mg mannitol/vial. This amount of mannitol might lead to some hazy solution upon reconstitution, which is generally to be avoided.

### Example 6: Composition #019

| Ingredients | mg/mL | | mg/vial | |
|---|---|---|---|---|
| Metolazone | 1 | mg | 10 | mg |
| Tertiary butyl alcohol | 348.7 | mg | 3487 | mg |
| (TBA-45.0%) $* | | | | |
| Mannitol (1%) | 10.0 | mg | 100 | mg |

| | | | |
|---|---|---|---|
| Water for injection* | Q.s to mL | Q.s to 10 | mL |

*Removed during lyophilization.
$: TBA density 0.775 g/mL used for calculation

The manufacturing process was like the previous examples.

Lyophilization Parameters

| | Temp. | Ramp | Hold | Vacuum |
|---|---|---|---|---|
| Stages | (° C.) | (min) | (min) | (mTorr) |
| Pre-freeze | 5 | — | — | — |
| Thermal Treatment | 5 | 45 | 90 | — |
| (Freezing) | −5 | 40 | 120 | — |
| | −35 | 120 | 200 | — |
| | −15 | 80 | 200 | |
| | −35 | 80 | 200 | |
| Evacuation (shelf | −35 | — | — | 180 |
| driven) | | | | |
| Primary drying | −35 | 10 | 30 | 180 |
| | −30 | 40 | 500 | 180 |
| | −20 | 160 | 1880 | 380 |
| | −20 | 1 | 90 | 380 |
| Secondary drying | 5 | 70 | 500 | 380 |
| | 25 | 60 | 360 | 220 |

To investigate reconstitution time, the following diluents were prepared at pH 7.5 with a target volume of 10 mL.

TABLE 3
Diluent composition PEG in Phosphate buffer

| Ingredients | g/mL | | g/vial | |
|---|---|---|---|---|
| Polyethylene glycol-400 (40.0%) | 0.452 | gms | 4.52 | gms |

| | | | |
|---|---|---|---|
| Phosphate buffer pH 7.5 | Q.s to mL | Q.s to 10 | mL |

Density of Polyethylene glycol-400 −1.13 g/mL

The batch was manufactured and filled into the 10 mL vials, which were stoppered and sealed. After sealing the vials were autoclaved at 121° C. for 15 minutes.

TABLE 4
Diluent composition PEG in TRIS buffer

| Ingredients | g/mL | | g/vial | |
|---|---|---|---|---|
| Polyethylene glycol-400 (40.0%) | 0.452 | gms | 4.52 | gms |

| | | | |
|---|---|---|---|
| TRIS buffer pH 7.5 | Q.s to mL | Q.s to 10 | mL |

The batch was manufactured and filled into the 10 mL vials, which were stoppered and sealed. After sealing the vials were autoclaved at 121° C. for 15 minutes.

TABLE 5
reconstitution with 10 mL diluents at pH 7.5

| | | Diluent | Diluent pH | pH of | Recon | |
|---|---|---|---|---|---|---|
| | | pH prior | post | recon | time | |
| Buffer | Description | autoclave | autoclave | product | (s) | Description |
| Phosphate | Clear | 7.348 | 6.712 | 6.69 | 120 | Clear |
| Buffer + 40% | solution | | | | | solution |
| PEG 400 v/v | | | | | | |
| TRIS buffer + | Clear | 8.608 | 8.047 | 8.028 | 120 | Clear |
| 40% PEG | solution | | | | | solution |
| 400 v/v | | | | | | |

Further experimentation of mannitol was done. The following trials exemplify the impact of 5 mg/mL and 15 mg/mL mannitol.

EXAMPLE 7
Composition: #20

| Ingredients | mg/mL | | mg/vial |
|---|---|---|---|
| Metolazone | 1 | mg | 10 |
| Tertiary butyl alcohol (TBA 50.0%) $* | 387.5 | mg | 3875 |
| Mannitol (1%) | 5.0 | mg | 50 |

| | | |
|---|---|---|
| Water for injection* | Q.s to mL | Q.s to 10 mL |

*Removed during lyophilization.
$: TBA density 0.775 g/mL used for calculation.

EXAMPLE 8
Composition: #21

| Ingredients | mg/mL | | mg/vial |
|---|---|---|---|
| Metolazone | 1 | mg | 10 |
| Tertiary butyl alcohol (TBA 50.0%) $* | 387.5 | mg | 3875 |
| Mannitol (1%) | 15.0 | mg | 150 |

| | | |
|---|---|---|
| Water for injection* | Q.s to mL | Q.s to 10 mL |

*Removed during lyophilization.
$: TBA density 0.775 g/mL used for calculation.

EXAMPLE 9
Composition: #16

| Ingredients | mg/mL | | mg/vial |
|---|---|---|---|
| Metolazone | 1 | mg | 10 |
| Tertiary butyl alcohol (TBA 50.0%) $* | 387.5 | mg | 3875 |
| Mannitol (1%) | 10.0 | mg | 100 |
| Kolliphor HS15(1.5%) | 15.0 | mg | 150 |

| | | |
|---|---|---|
| Water for injection | Q.s to mL | Q.s to 10 mL |

*Removed during lyophilization.
$: TBA density 0.775 g/mL used for calculation.

Examples 7-9 were lyophilized using the parameters of Example 6 and were tested for reconstitution and clarity using diluents from Table 5 and resulted in a clear solution within 120 seconds.

### Example 10: Composition: #24

| Ingredients | mg/mL | | mg/vial | |
|---|---|---|---|---|
| Metolazone | 1 | mg | 10 | mg |
| Tertiary butyl alcohol (TBA 50.0%) $* | 387.5 | mg | 3875 | mg |
| Mannitol (0.5%) | 5.0 | mg | 50 | mg |
| Kolliphor HS-15 (0.4%) | 4.0 | mg | 40 | mg |

| | | | |
|---|---|---|---|
| Water for injection* | Q.s to mL | Q.s to 10 | mL |

*Removed during lyophilization.
$: TBA density 0.775 g/mL used for calculation.

### Example 11: Composition #26

| Ingredients | mg/mL | | mg/vial | |
|---|---|---|---|---|
| Metolazone | 1 | mg | 10 | mg |
| Tertiary butyl alcohol (TBA 50.0%) $* | 387.5 | mg | 3875 | mg |
| Mannitol (0.25%) | 2.5 | mg | 25 | mg |

| | | | |
|---|---|---|---|
| Water for injection | Q.s to mL | Q.s to 10 | mL |

*Removed during lyophilization.
$: TBA density 0.775 g/mL used for calculation.

Examples 10-11 were lyophilized using the parameters of Example 6 and were tested for reconstitution as described below.

Reconstitution results of batch No: Example 10 and 11

| | | | pH of | |
|---|---|---|---|---|
| Comp. | | Reconstitution | Reconstituted | |
| No. | Diluent Used | time [seconds] | solution | Observations |
| #24 | 40% v/v PEG-400 in | 120 | 7.861 | Clear Colorless solution |
| | phosphate buffer pH 7.5 | | | |
| | [Autoclaved] | | | |
| #24 | (40% v/v PEG-400 in | 120 | 6.313 | Clear Colorless solution |
| | TRIS buffer pH 7.5 | | | |
| | [Autoclaved] | | | |
| #26 | 40% v/v PEG-400 in | 120 | 7.893 | Clear Colorless solution |
| | phosphate buffer pH 7.5 | | | |
| | [Autoclaved] | | | |
| #26 | 40% v/v PEG-400 in | 120 | 6.305 | Clear Colorless solution |
| | TRIS buffer pH 7.5 | | | |
| | [Autoclaved] | | | |

### Example 12: Effect of Mannitol Concentration

Three batches with different levels of mannitol were prepared and drug product quality and performance attributes were tested.

| Sample No. | 030 | 042B | 042A |
|---|---|---|---|
| Ingredients | mg/mL | mg/mL | mg/mL |
| Metolazone | 1.00 | 1.00 | 1.00 |
| Tertiary butyl alcohol | 271.25 | 271.25 | 271.25 |
| (TBA 35.0%) $ | | | |
| Mannitol | 5.00 | 25.00 | 50.00 |
| Water for injection | Q.s to mL | Q.s to mL | Q.s to mL |

A*Water for injection will be removed during lyophilization.
$: TBA density 0.775 g/mL used for calculation.

Procedure:
1. Dispense the batch quantities of metolazone and all raw materials using weighing balance. Thaw TBA prior to dispensing.
2. Collect slightly excess Water for Injection (WFI) of total batch size in a clean SS316L jacketed vessel and hold at room temperature.
3. In about 20% v/v of Water for Injection (WFI) of total batch size, add 100% w/v of batch quantity of tertiary butyl alcohol (TBA) on weight basis based on the density and mix well to get a uniform mixture.
4. Add metolazone to the solution TBA and WFI and dissolve by stirring at 500-800 RPM till clear solution obtained using overhead stirrer.
5. Add mannitol to the solution in Step 4 and dissolve by using overhead stirring. Once clear solution is observed, make up the volume up to 100% v/v using WFI.
6. Filter the solution from using 0.22 μm polyether sulfone (PES) or other suitable filter membrane with filtration kit using Nitrogen gas.
7. Fill at target volume of filtered solution into the 10 mL USP Type I, glass vial and partially stopper the vials with 20 mm Lyo rubber stoppers.
8. Load the partially stoppered vials into a lyophilizer and lyophilize the product under nitrogen environment using a lyophilization process as described above.

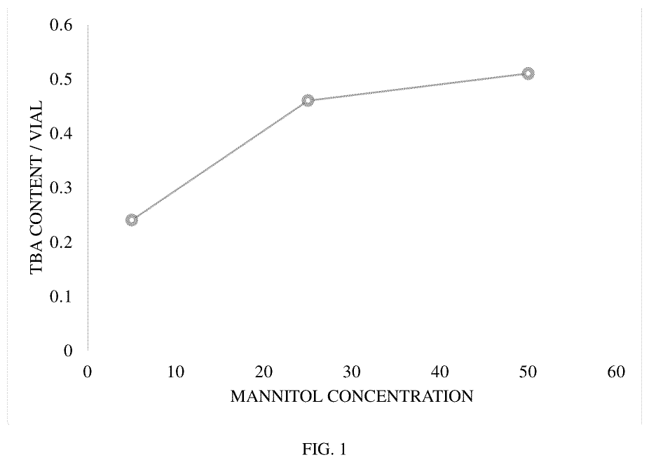

The residual TBA content of the batches with different mannitol concentration was determined and is tabulated below and graphed in FIG. 1.

Effect of Mannitol in Residual TBA Level

| | Mannitol | TBA Level |
|---|---|---|
| Batch No | mg/mL | (mg/vial) |
| ROY04-FD/189-042A | 50 | 0.51 |
| ROY04-FD/189-042B | 25 | 0.46 |
| ROY04-FD/189-030 | 5 | 0.24 |

Observations: as seen in FIG. 1, the higher the mannitol concentration, the higher the residual TBA content observed in the vials. Based on the ICH (International Council for Harmonization), 1700 ppm or 1.75 mg/vial is the limit for residual TBA. The above test results that a residual TBA level from 0.2 mg to 0.5 mg/vial depending on mannitol concentration, which is well within the target limit as per ICH.

Impurity B: A major acid and base catalyzed hydrolytic degradation product of metolazone is a metolazone benzamide analog, 2-amino-4-chloro-5-sulfamoyl-N-(o-tolyl)benzamide (Impurity-B). It was determined that 1 week at 60° C. provides levels equal to 3 months (3 M) at 40° C./75% RH (relative humidity). Therefore, 1 week at 60° C. was used as a surrogate condition to determine impurity levels in the test products.

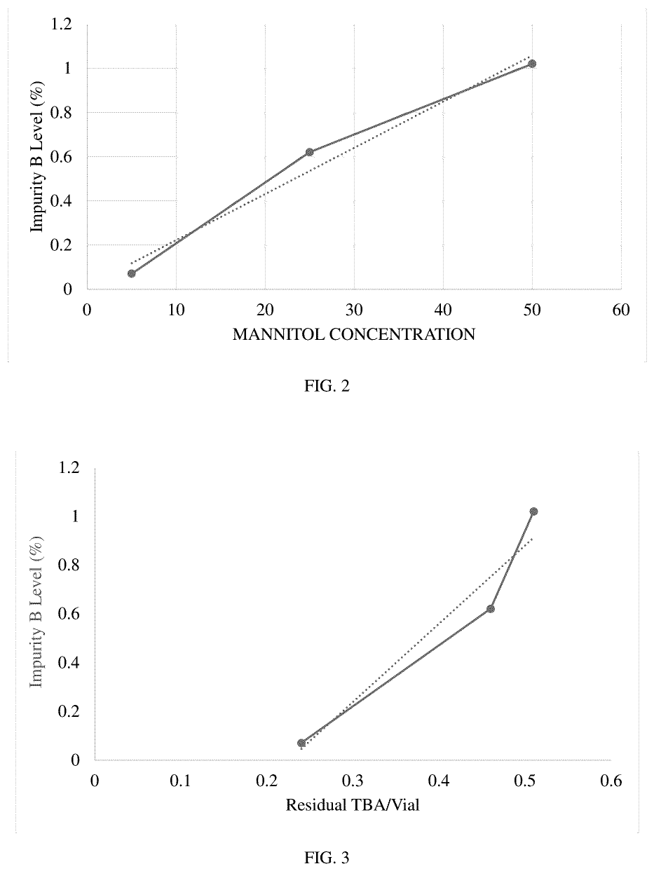

Impurity B content from 1 week at 60° C. of batches made with different mannitol concentrations are shown in the table below and graphed in FIG. 2 and FIG. 3.

Effect of Mannitol on Impurity B

| Batch No | Mannitol mg/mL | Imp B (%) |
|---|---|---|
| ROY04-FD/189-042A | 50 | 1.02 |
| ROY04-FD/189-042B | 25 | 0.62 |
| ROY04-FD/189-030 | 5 | 0.07 |

Observations:
a. The results above (also see FIG. 2) show that Impurity B generation in the formulation is directly proportional to the mannitol concentration. The slope of this relationship is 0.2062 (±10%) or 0.18558 to 0.22682. This slope can be utilized to select the optimal mannitol concentration for formulations.
b. The results above (also see FIG. 3) show that Impurity B generation in formulation is directly proportional to residual TBA concentration. The slope of this relationship is 3.2108 (±10%) or 2.88972 to 3.53188. This slope can be utilized to select optimal residual TBA concentration for formulations.

### Example 13: Effect of TBA/Water Ratio

Saturation solubility study of metolazone was conducted in different water to tertiary butyl alcohol ratios ranging from 20-40% of TBA and 80-60% water. The study was conducted in triplicate. The saturation solubility data suggests that a minimum of 30% of TBA is required to solubilize metolazone concentration of one mg/mL.

| | | Observation | | |
|---|---|---|---|---|
| | | Solubility of | | Average |
| Batch | | each trial | | solubility |
| Number | Details | (mg/mL) | | (mg/mL) |
| 028A | Saturation solubility with 20% | Vial-1 | 0.267 | 0.279 |
| | Tertiary butyl alcohol and 80% | Vial-2 | 0.289 | |
| | water mixture [20:80] | Vial-3 | 0.281 | |
| 028B | Saturation solubility with 30% | Vial-1 | 0.948 | 1.014 |
| | Tertiary butyl alcohol and 70% | Vial-2 | 1.044 | |
| | water mixture [30:70] | Vial-3 | 1.049 | |
| 028C | Saturation solubility with 40% | Vial-1 | 2.171 | 2.106 |
| | Tertiary butyl alcohol and 60% | Vial-2 | 2.122 | |
| | water mixture [40:60] | Vial-3 | 2.025 | |

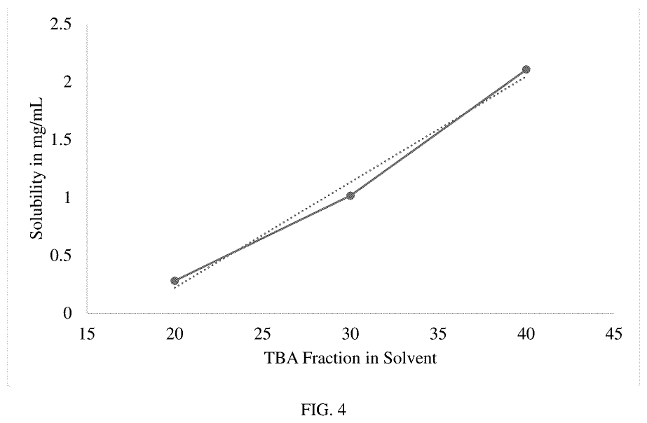

Observations:
a. TBA is required in combination with water to dissolve metolazone.
b. Solvent (TBA:water) in the ratio of 30:70 to 40:60 can solubilize>1 mg/mL metolazone.
c. As the TBA content increases, the solubility of metolazone increases (see FIG. 4). The result shows that metolazone solubility is directly proportional to the TBA fraction in solvent. The slope of this relationship is 0.0913 (±10%) or 0.08217 to 0.10043. This slope can be utilized to select optimal TBA concentration to solubilize metolazone for formulations.

### Example 14: Effect of Diluent

The diluent selection study was conducted to select the best diluent combination for metolazone. The following components were evaluated in combination with phosphate buffer to check the solubility of metolazone and to observe the solution clarity.

Procedure for Example no ITT-1 to 111-5: An excess amount of drug substance PSD (particle size distribution) 10 micron (60 mg) was added to the 20 mL diluent mixtures specified in Ex. III-1 to II-5 so that the theoretical concentration was 3 mg/mL. After drug addition, these samples were sonicated up to 4 hr using a sonicator at room temperature. Solution clarity and quantitative solubility were observed.

| Study | | | |
|---|---|---|---|
| Example | Study details | Composition | Observations |
| Ex: III-1 | 031A | 40% PEG-400 + Phosphate | Solubility |
| | PEG-400 + | buffer pH 7.5, QS to 20 mL | (mg/mL): |
| | Phosphate | Drug concentration: | 1.862 |
| | buffer | 3 mg/mL (60 mg/20 mL) | |
| | pH 7.5 | | |
| Ex: III-2 | 031B | 40% PEG-400 + Tris buffer | Solubility |
| | PEG-400 + | pH 7.5, QS to 20 mL | (mg/mL): |
| | Tris buffer | Drug concentration: | 1.284 |
| | pH 7.5 | 3 mg/mL (60 mg/20 mL) | |
| Ex: III-3 | 031C | 40% PEG-400 + Kolliphor | Solubility |
| | PEG-400 + | HS-15 0.4% + | (mg/mL): |
| | Kolliphor | Phosphate buffer | 2.163 |
| | HS-15 + | pH 7.5, QS to 20 mL | |
| | Phosphate | Drug concentration: | |
| | buffer pH 7.5 | 3 mg/mL (60 mg/20 mL) | |
| Ex: III-4 | 031D | 40% PEG-400 + Kolliphor | Solubility |
| | PEG-400 + | HS-15 0.4% + | (mg/mL): |
| | Kolliphor | Tris buffer pH 7.5, | 2.306 |
| | HS-15 + | QS to 20 mL | |
| | Tris buffer | Drug concentration: | |
| | pH 7.5 | 3 mg/mL (60 mg/20 mL) | |
| Ex: III-5 | 031E | 50% Tertiary butyl alcohol | Solubility |
| | TBA and | and 50% Water for injection | (mg/mL): |
| | Water | Drug concentration: | 3.125 |
| | | 3 mg/mL (60 mg/20 mL) | |

Observations: Metolazone can be solubilized in any of the following mixtures with an appropriately selected concentration.
a. PEG-400+Phosphate buffer pH 7.5;
b. PEG-400+Tris buffer pH 7.5;
c. PEG-400+Kolliphor HS-15+Phosphate buffer pH 7.5;
d. PEG-400+Kolliphor HS-15+Tris buffer pH 7.5; and,
e. TBA and Water.

The order of quantitative solubility is:
a. TBA and Water>
b. PEG-400+Kolliphor HS-15+Tris buffer pH 7.5>
c. PEG-400+Kolliphor HS-15+Phosphate buffer pH 7.5>
d. PEG-400+Phosphate buffer pH 7.5>
e. PEG-400+Tris buffer pH 7.5.

Observations on solubility: with the use of 40% PEG in all tested buffers, a >1.2 mg/mL solubility of Metolazone was obtained for the metolazone API with particle size D90<10 microns. This provides a baseline solubility for Form I crystalline API. However, after lyophilization, it was expected that the drug will be lyophilized to a submicron level either amorphous or crystalline or a combination of both, leading to significant improvement in solubility of API.

The solubility of the lyophilized metolazone was carried out by removing the lyophilized powder from the vial and adding it to 5 mL of Diluent (PEG-400 (40%)+Phosphate buffer pH 7.5). Three (3) vials of lyophilized metolazone powder cake, equivalent to 90 mg total weight of the powder containing 15 mg of metolazone, were completely solubilized in 5 mL of diluent. This is at least 3 mg/mL solubility of metolazone. It was also possible to add more vials into the 5 mL diluent with possibility of achieving solubility of 20 mg metolazone in 5 mL of diluent, resulting in 4 mg/mL metolazone solubility. This is a 400% increase in solubility of metolazone (1.8 mg/mL without lyophilization versus 4 mg/mL after lyophilization).

| Vials added to diluent | Resulting Solution |
|---|---|
| 1 vial (30 mg powder\hat{ }) | Clear solution |
| 1 vial (30 mg powder) = 60 mg | Clear solution |
| 1 vial (30 mg powder) = 90 mg | Clear solution |
| 1 vial (30 mg powder) = 120 mg | Clear solution |

The following tables of PEG-400 to drug ratios (weight/weight) are part of the present invention. \hat{ }Lyophilised metolazone+mannitol powder containing 5 mg metolazone/vial in 30 mg

TABLE A
10% PEG 400 in Phosphate buffer pH 7.5, 5 mL Diluent

| Drug | | Ratio of | |
|---|---|---|---|
| Strength | PEG 400 | Drug to | Ratio of PEG |
| mg/vial | (microgram) | PEG 400 | 400 to Drug |
| 1 | 500 | 0.002 | 500 |
| 5 | | 0.01 | 100 |
| 10 | | 0.02 | 50 |
| 15 | | 0.03 | 33.3 |
| 20 | | 0.04 | 25 |

TABLE B
15% PEG 400 in Phosphate buffer pH 7.5, 5 mL Diluent

| Drug | | Ratio of | |
|---|---|---|---|
| Strength | PEG 400 | Drug to | Ratio of PEG |
| mg/vial | (microgram) | PEG 400 | 400 to Drug |
| 1 | 750 | 0.0013 | 750 |
| 5 | | 0.0067 | 150 |
| 10 | | 0.0133 | 75 |
| 15 | | 0.0200 | 50 |
| 20 | | 0.0267 | 37.5 |

TABLE C
20% PEG 400 in Phosphate buffer pH 7.5, 5 mL Diluent

| Drug | | Ratio of | |
|---|---|---|---|
| Strength | PEG 400 | Drug to | Ratio of PEG |
| mg/vial | (microgram) | PEG 400 | 400 to Drug |
| 1 | 1000 | 0.0010 | 1000 |
| 5 | | 0.0050 | 200 |
| 10 | | 0.0100 | 100 |
| 15 | | 0.0150 | 66.7 |
| 20 | | 0.0200 | 50 |

TABLE D
30% PEG 400 in Phosphate buffer pH 7.5, 5 mL Diluent

| Drug | | Ratio of | |
|---|---|---|---|
| Strength | PEG 400 | Drug to | Ratio of PEG |
| mg/vial | (microgram) | PEG 400 | 400 to Drug |
| 1 | 1500 | 0.0007 | 1500 |
| 5 | | 0.0033 | 300 |
| 10 | | 0.0067 | 150 |
| 15 | | 0.0100 | 100 |
| 20 | | 0.0133 | 75 |

TABLE E
40% PEG 400 in Phosphate buffer pH 7.5, 5 mL Diluent

| Drug | | Ratio of | |
|---|---|---|---|
| Strength | PEG 400 | Drug to | Ratio of PEG |
| mg/vial | (microgram) | PEG 400 | 400 to Drug |
| 1 | 2000 | 0.0005 | 2000 |
| 5 | | 0.0025 | 400 |
| 10 | | 0.0050 | 200 |
| 15 | | 0.0075 | 133.3 |
| 20 | | 0.0100 | 100 |

### Example 15: Effect of the Lowest PEG 400 in Diluent

Diluent volume and % of PEG are important to achieve clear solutions for reconstituted metolazone solutions. Solution clarity with the diluent containing 40% PEG-400 was shown above to be useful. The following investigation was carried out to identify the lowest % of PEG-400 that can achieve a clear solution when it is combined with phosphate buffer at pH 7.5.

Lyophilized metolazone batches were prepared containing different % of PEG 400 such as 10%-15% with phosphate buffer at pH 7.5. These diluents were used for reconstitution studies and the following observation was obtained.

| | | Reconstitution | |
|---|---|---|---|
| Batch number | Details of diluents | Time | Clarity |
| 034 | Diluent Batch No.: 041A | Reconstitution | Clear |
| (Formulation | Details: 10% PEG-400 in | time <120 | Colorless |
| containing 50.0% | phosphate buffer pH 7.5. | seconds. | solution |
| TBA:50.0% Water | Diluent Batch No.: 041B | Reconstitution | Clear |
| with 5 mg/mL | Details: 15% PEG-400 in | time <120 | Colorless |
| mannitol | phosphate buffer pH 7.5. | seconds. | solution |
| concentration— | Buffer Capacity: 0.05M | | |
| 5 mL fill | | | |
| Lyophilized to form | | | |
| a porous cake | | | |
| 039 | Diluent Batch No.: 041A | Reconstitution | Clear |
| (Formulation | Details: 10% PEG-400 in | time <120 | colorless |
| containing 35.0% | phosphate buffer pH 7.5. | seconds. | solution |
| TBA:65.0% Water | Diluent Batch No.: 041B | Reconstitution | Clear |
| with 5 mg/mL | Details: 15% PEG-400 in | time <120 | Colorless |
| mannitol | phosphate buffer pH 7.5. | seconds | solution |
| concentration— | | | |
| 5 mL fill | | | |
| Lyophilised to form | | | |
| a porous cake | | | |

Observations: PEG 400 in the concentration of 10% w/v or above in combination and Phosphate buffer or any of the solvent mixture alone or combination (as listed in Ex-3) can be used to reconstitute the metolazone lyophilized injection.

The following tables of drug to diluent drug ratios (weight/volume) are part of the present invention.

TABLE A
Drug content to Diluent Ratios

| Drug Content | Diluent | Ratio of Drug to | Ratio of |
|---|---|---|---|
| (mg/per Vial) | Volume (mL) | Diluent | Diluent to Drug |
| 1 | 5 | 0.2 | 5 |
| 5 | | 1 | 1 |
| 10 | | 2 | 0.5 |
| 15 | | 3 | 0.33 |
| 20 | | 4 | 0.25 |

TABLE B
Drug content to Diluent Ratios

| Drug Content | Diluent | Ratio of Drug to | Ratio of Diluent |
|---|---|---|---|
| (mg/per Vial) | (mL) | Diluent | to Drug |
| 1 | 10 | 0.1 | 10 |
| 5 | | 0.5 | 2 |
| 10 | | 1 | 1 |
| 15 | | 1.5 | 0.67 |
| 20 | | 2 | 0.5 |

TABLE C
Drug content to Diluent Ratios

| Drug Content | Diluent | Ratio of Drug to | Ratio of Diluent to |
|---|---|---|---|
| (mg/per Vial) | (mL) | Diluent | Drug |
| 1 | 15 | 0.07 | 15.00 |
| 5 | | 0.3 | 3.00 |
| 10 | | 0.7 | 1.50 |
| 15 | | 1.0 | 1.00 |
| 20 | | 1.3 | 0.75 |

TABLE D
Drug content to Diluent Ratios

| Drug Content | Diluent | Ratio of Drug to | Ratio of Diluent to |
|---|---|---|---|
| (mg/per Vial) | (mL) | Diluent | Drug |
| 1 | 20 | 0.05 | 20 |
| 5 | | 0.3 | 4 |
| 10 | | 0.5 | 2 |
| 15 | | 0.8 | 1.33 |
| 20 | | 1 | 1 |

Surprisingly it was found that, metolazone can effectively be lyophilized using an aqueous or hydroalcoholic system which can further be reconstituted with a suitable diluent containing a buffer and solubilizer.

For renal impaired patients where metolazone is expected to be used, it's important that the volume of fluids that are administered are minimal, with the above-identified compositions of metolazone, it is possible to reconstitute lyophilized metolazone with 10-20 mL or even less than 10 mL reconstitution fluid (diluent). The reconstituted fluid can be administered directly to a patient in need and if needed be diluted with different infusion solvents before administration.

All references listed herein are individually incorporated herein in their entirety by reference.

Numerous modifications and variations of the invention are possible considering the above teachings. It is therefore to be understood that within the scope of the appended claims, the invention may be practiced otherwise than as specifically described herein.

1. A metolazone lyophilizate composition, comprising:
a. metolazone; and,
b. a bulking agent;
wherein
the metolazone lyophilizate composition is in the form of a lyophilized powder; and,
the bulking agent is selected from mannitol, lactose, dextrose, sucrose and hydroxypropyl-β-cyclodextrin.

2. The lyophilizate composition of claim 1, wherein the composition, comprises:
a. about 2.5-20 mg metolazone; and,
b. about 2.5-1000 mg of mannitol.

3. The lyophilizate composition of claim 1, wherein the composition, comprises:
a. about 2.5-10 mg metolazone; and,
b. about 2.5-500 mg of mannitol.

4. The lyophilizate composition of claim 1, wherein the composition, comprises:
a. about 5 mg metolazone; and,
b. about 25-55 mg of mannitol.

5. The lyophilizate composition of claim 1, wherein the composition, comprises:
a. about 10 mg metolazone; and,
b. about 50-150 mg of mannitol.

6. The lyophilizate composition of claim 1, wherein the composition is housed in a container.

7. The lyophilizate composition of claim 1, wherein the composition contains less than 1.75 mg of tertiary butyl alcohol.

8. A liquid, metolazone pre-lyophilizate composition, comprising:
a. metolazone;
b. a bulking agent;
c. an alcohol; and,
d. water;
wherein:
the liquid, metolazone pre-lyophilizate composition forms a lyophilized powder after being subjected to lyophilization; and,
the bulking agent is selected from mannitol, lactose, dextrose, sucrose and hydroxypropyl-β-cyclodextrin.

9. The metolazone pre-lyophilizate composition of claim 8, comprising:
a. about 0.1-2 mg/ml metolazone;
b. about 1-50 mg/mL mannitol;
c. about 30-70% v/v tertiary butyl alcohol; and,
d. the remainder, water.

10. The metolazone pre-lyophilizate composition of claim 8, comprising:
a. about 0.1-1.5 mg/ml metolazone;
b. about 2.5-60 mg/mL mannitol;
c. about 30-70% by volume tertiary butyl alcohol; and,
d. the remainder, water.

11. The metolazone pre-lyophilizate composition of claim 8, comprising:
a. about 0.1-1 mg/ml metolazone;
b. about 5-10 mg/mL mannitol;
c. about 40-60% by volume tertiary butyl alcohol; and,
d. the remainder, water.

12. The metolazone pre-lyophilizate composition of claim 8, further comprising:
about 1-15 mg/mL macrogol-15-hydroxystearate.

13. A metolazone lyophilizate kit, comprising:
a. a first container, comprising: a metolazone lyophilizate composition in the form of a lyophilized powder, comprising:
i. metolazone; and,
ii. a bulking agent; and,
b. a second container, comprising: a pharmaceutically acceptable diluent, comprising:
i. a solubilizer;
ii. a buffer; and,
iii. water;
wherein the diluent is suitable to reconstitute the lyophilizate composition into a drug product having a pH of about 5.0-8.0 for parenteral administration.

14. The metolazone lyophilizate kit of claim 13, further comprising a connect for holding the first and second containers.

15. The metolazone lyophilizate kit of claim 13, wherein the diluent, further comprises:
about 2-4% w/v macrogol-15-hydroxystearate.

16. The metolazone lyophilizate kit of claim 13, wherein
the metolazone lyophilizate composition, comprises:
i. about 2.5-20 mg metolazone; and,
ii. about 2.5-1000 mg of mannitol; and,
the diluent, comprises:
i. about 25-45% w/v polyethylene glycol 400;
ii. TRIS buffer or phosphate buffer; and,
iii. the remainder, water;
wherein the pH of the diluent is about 7-8.

17. The metolazone lyophilizate kit of claim 13, wherein
the metolazone lyophilizate composition, comprises:
i. about 2.5-10 mg metolazone; and,
ii. about 25-500 mg of mannitol;
the diluent, comprises:
i. about 30-40% w/v polyethylene glycol 400;
ii. TRIS buffer or phosphate buffer; and,
iii. the remainder, water;
wherein the pH of the diluent is about 7.5.

18. The metolazone lyophilizate kit of claim 13, wherein the volume of the diluent is about 5-20 mL.

19. The metolazone lyophilizate kit of claim 13, wherein the metolazone lyophilizate composition contains less than 1.75 mg of tertiary butyl alcohol.

20. A process for reconstituting a metolazone lyophilizate composition, the process comprising:
contacting the metolazone lyophilizate composition and a pharmaceutically acceptable diluent of claim 13; and,
mixing the resulting solution until clear;
wherein the resulting clear solution is suitable for parenteral administration.

21. The process of claim 20, wherein a clear solution is obtained in less than about 5 minutes.

22. A method of treating a patient in need of a natriuretic and/or diuretic treatment, comprising:
contacting the metolazone lyophilizate composition and pharmaceutically acceptable diluent of claim 13;
mixing the resulting solution until a clear, reconstituted metolazone solution is obtained;
administering to the patient in need of the natriuretic and/or diuretic treatment a therapeutically effective amount of the clear, reconstituted metolazone solution.

23. A method of treating a patient with furosemide resistance, comprising:
contacting the metolazone lyophilizate composition and pharmaceutically acceptable diluent of claim 13;
mixing the resulting solution until a clear, reconstituted metolazone solution is obtained;
administering to the patient in need of furosemide resistance treatment a therapeutically effective amount of the clear, reconstituted metolazone solution.
